(19) United States
(12) Patent Application Publication
(10) Pub. No.: US 2017/0080202 A1
(43) Pub. Date: Mar. 23, 2017

(54) Catheter Line with Lock Fitting

(71) Applicants: Michael K. Justus, (US); Jesse Michael Justus, Sheboygan, WI (US)

(72) Inventor: Michael K. Justus, Beloit, WI (US)

(73) Assignees: Michael K. Justus, Beloit, WI (US); Jesse Michael Justus, Sheboygan, WI (US)

(21) Appl. No.: 15/268,632

(22) Filed: Sep. 18, 2016

Related U.S. Application Data
(60) Provisional application No. 62/220,948, filed on Sep. 18, 2015.
(60) Provisional application No. 62/220,767, filed on Sep. 18, 2015.

(51) Int. Cl. A61M 39/10; A61M 25/00

(52) U.S. Cl. CPC A61M 39/1011; A61M 25/0045

(57) ABSTRACT

A catheter assembly cooperates with a catheter lock enclosure having a base with an entry slot in an end wall. The catheter assembly has a flexible injection line extending from a base and an exterior tube surrounding the injection line and extending from the base. An exterior barrier extends radially outwardly from the exterior tube, and an interior barrier extends radially outwardly from the injection line and is spaced axially along the injection line from the exterior barrier. A gap is defined between the interior barrier and the exterior barrier, which receives the lock enclosure bottom part end wall to restrain the position of the catheter assembly with respect to the lock enclosure, and to restrict access to an interior of the catheter lock enclosure. The exterior tube and the interior and exterior barriers may be formed of high density polyethylene, or hard urethane, or Ultra-high-molecular-weight polyethylene (UHMW).

### CROSS REFERENCES TO RELATED APPLICATIONS

This application claims the benefit of priority of U.S. provisional patent App. No. 62/220,948, filed Sep. 18, 2015, the disclosure of which is incorporated by reference herein.

### STATEMENT AS TO RIGHTS TO INVENTIONS MADE UNDER FEDERALLY SPONSORED RESEARCH AND DEVELOPMENT

Not applicable.

### BACKGROUND OF THE INVENTION

The present invention relates to catheters which cooperate with lock-out devices to prevent unauthorized access by intravenous drug abusers.

Abusers of intravenous drugs such as heroin may suffer from medical conditions requiring insertion of catheters. Be it the treatment of infections by regular intravenous injection of antibiotics, or amelioration of kidney malfunctions by regular dialysis, the drug abuser may be provided with an intravenous peripherally inserted central catheter or PICC line or other catheter arrangement. This catheter, while usually inserted in the hospital, can remain in place for weeks or months, and makes it a simple matter to rapidly inject necessary fluids without difficulty in finding a vein. This ease of intravenous access, however, can be a serious hazard for outpatients who are habitual drug abusers, who can take advantage of this catheter to self-administer unprescribed quantities of narcotics or other controlled substances. Providing a drug abuser with an unsupervised catheter for outpatient care can lead to drug overdose and death. To protect these high risk patients, who might otherwise not need full-time nursing care, they must be hospitalized and retained under supervision for as long as the catheter is in place. Hospitalization is vastly more costly than outpatient care, and represents a less than optimal utilization of health care resources.

U.S. Pat. No. 8,858,505, entitled Catheter Injection Port Lock, discloses a tamper-evident lock for securing access to the injection port of a catheter. The lock has two plastic parts which slide together and which define an interior enclosure which receives a catheter line injection port. The housing halves have overlying flanges, through which a fastener extends to secure the device in a closed configuration. The fastener interacts with the flanges in such a way that it cannot be removed without distorting the device and giving evidence of tampering. Should the patient remove the box to defeat its restrictions, the damage to the box provides ready evidence that the patient has tampered with the device. Also disclosed is a zippered sleeve assembly for protecting the catheter line as it extends between the inlet port and the base.

The U.S. Pat. No. 8,858,505 patent sleeve permits the lock to work with a wide variety of catheters. Yet a specially constructed catheter configured for use with an injection port would offer advantages of lower cost and greater ease of use.

### SUMMARY OF THE INVENTION

A catheter assembly cooperates with a catheter lock enclosure having a bottom part with an entry slot in an end wall. The catheter assembly has a flexible injection line extending from a picc line base and an puncture resistant exterior tube surrounding the injection line and extending from the picc line base. An exterior barrier extends radially outwardly from the puncture resistant exterior tube, and an interior barrier extends radially outwardly from the injection line and is spaced axially along the injection line from the exterior barrier. A gap is defined between the interior barrier and the exterior barrier, which receives the lock enclosure bottom part end wall to restrain the position of the catheter assembly with respect to the lock enclosure, and to restrict access to an interior of the catheter lock enclosure. The exterior tube and the interior and exterior barriers may be formed, by way of examples, of high density polyethelene, or hard urethane, or Ultra-high-molecular-weight polyethylene (UHMW).

It is an object of the present invention to provide a catheter protective sleeve which is provided as part of the catheter itself for convenient interaction with an injection port lock.

Further objects, features and advantages of the invention will be apparent from the following detailed description when taken in conjunction with the accompanying drawings.

### BRIEF DESCRIPTION OF THE DRAWINGS

FIG. 1 is a schematic view of a catheter assembly of the present invention attached to a patient with a catheter lock.

FIG. 2 is an exploded isometric view, partially broken away, of the catheter assembly and catheter lock of FIG. 1.

FIG. 3 is a side elevational view of the catheter assembly and injection port of FIG. 2.

FIG. 4 is a side elevational view, partially broken away in section of the catheter assembly and injection port of FIG. 3 secured within a catheter lock.

FIG. 5 is a cross sectional view of the arrangement of FIG. 4, taken along section line 4-4.

FIG. 6 is a fragmentary perspective view of an alternative embodiment catheter assembly of this invention.

FIG. 7 is a fragmentary side elevational view of the catheter assembly of FIG. 6 shown received within a catheter lock shown in cross-section.

FIG. 8 is a fragmentary side elevational view of another alternative embodiment catheter assembly of this invention in which the exterior tube, interior barrier, and exterior barrier are integrally formed.

### DESCRIPTION OF THE PREFERRED EMBODIMENTS

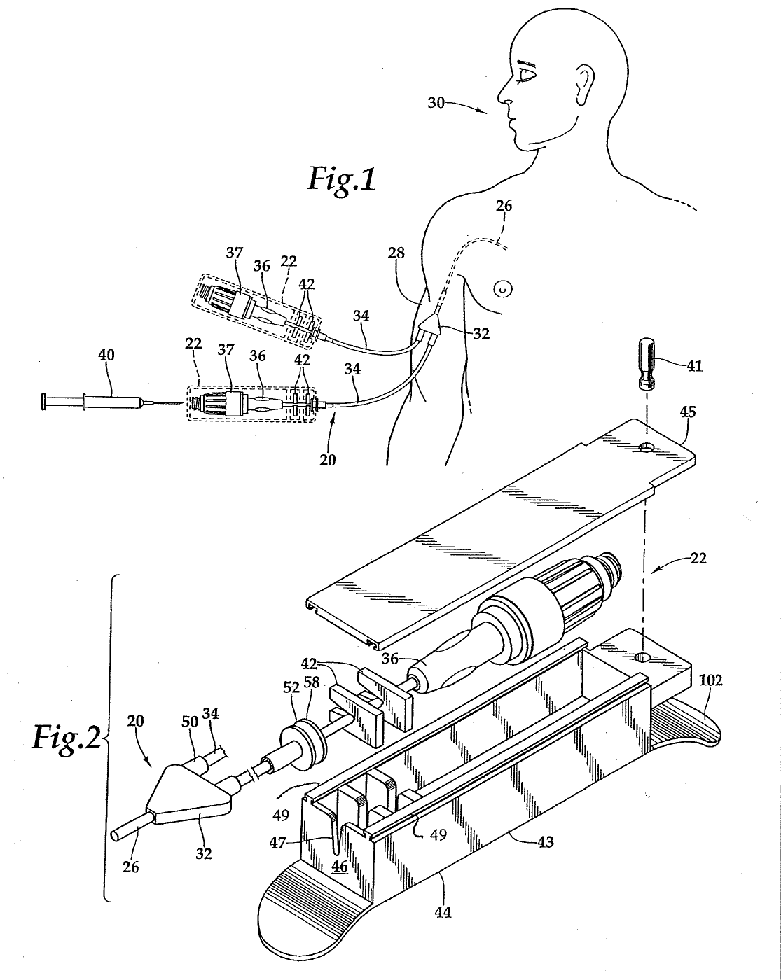

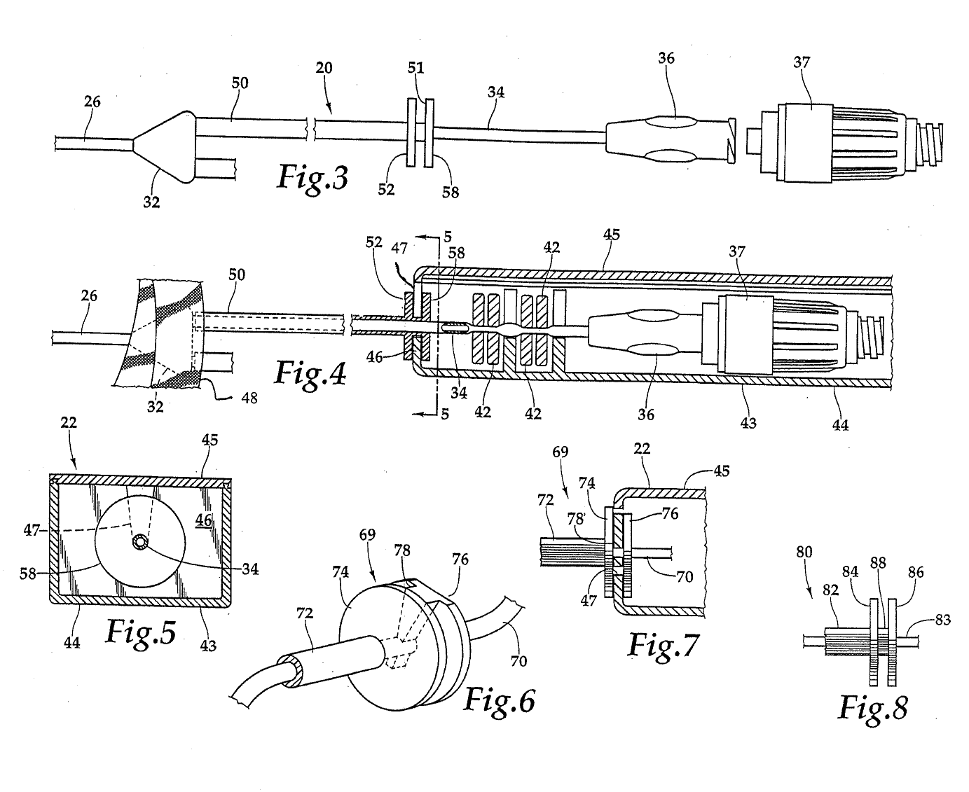

Referring more particularly to FIGS. 1-8, wherein like numbers refer to similar parts, a catheter assembly 20 is shown in FIGS. 1-5 for use with a tamper evident catheter lock 22. The catheter assembly 20 is configured to work with a catheter lock 22 such as the ones disclosed in U.S. Pat. No. 8,858,505, entitled Catheter Injection Port Lock, the disclosure of which is incorporated by reference herein. As shown in FIG. 1, the catheter assembly 20 has a flexible catheter 26 which is inserted into an upper extremity 28 of the patient 30, for example an arm. The catheter assembly 20 will typically have a pick line base 32 from which the catheter emerges and enters the patient through the skin. The picc line base is positioned to hold the catheter 26 against movement by attached to the patient for example of tape 48 as shown in FIG. 4. One or more injection lines 34 extends from the picc line base, each of which is terminated by an inlet port 36. The inlet ports 36 provide entryways to the injection lines 34 for delivery of fluids from an injection source such as a syringe 40 or a total parenteral nutrition bag or whatever injectable the patient requires. As shown in FIG. 3, the inlet ports may be provided with a positive displacement connector 37, such as the MaxPlus® clear needleless connector made by Carefusion Corporation of San Diego, Calif., and such as disclosed in U.S. Pat. No. 5,730,418, the disclosure of which is incorporated by reference herein. The lock 22 provides a mechanism for blocking access to the inlet ports 36 by the patient. The injection lines 34 may be provided with one or more mechanical clamps 42 which serve to prevent the escape or entry of liquids into the lines unless the clamps 42 are removed.

As shown in FIG. 2, the catheter assembly 20 interacts with the lock 22 to prevent access by the patient to the injection lines 34 between the picc line base 32 and the lock. The lock 22 has a housing 43 comprised of a bottom part 44 which receives a sliding lid 45 which together with the bottom part from locking ways 49 e.g., dovetail ways as shown, which hold the lid to the bottom part. After the catheter assembly injection line and injection port are received within the housing, the lid 45 is secured to the housing bottom part 44 with a connector 41 to close the housing in such a way that it cannot be opened without giving evidence of tampering, see further examples of securing the housing as shown in U.S. Pat. No. 5,730,418. An end wall 46 of the housing bottom part 44 has a entry slot 47 which may be V-shaped as best shown in FIG. 2. If the injection line 34 is not incased in a stiff puncture resistant exterior tube 50 as it passes through the V-shaped entry slot 47, the slot may serves as a clamp for the injection line.

The catheter assembly 20 has a puncture resistant exterior tube plastic exterior tube 50 which surrounds the injection line 34 and extends at least to an exterior barrier 52 which is puncture resistant and preferably stiff, and may be ring shaped or any shape which overlies and blocks at least most of the entry slot 47 formed in the end wall 46 of the lock housing 43. An interior barrier 58 extends from the injection line 34 or the exterior tube 50, and is closely spaced from the exterior barrier 52. The interior barrier 58 may also be formed of inflexible plastic, such that the interior barrier 58 can not be pulled through the entry slot 47, and may be of a similar shape to the exterior barrier 52. The exterior and interior barriers 52, 58 are spaced from one another along the injection line 34 by approximately the thickness of the housing end wall 46 e.g., the end wall thickness ±0-5%, 5-10%, or 10-20% of the end wall thickness. The exterior tube 50 and the interior and exterior barriers 58, 52 may be formed, by way of example, of high density polyethylene, or hard urethane, or Ultra-high-molecular-weight polyethylene (UHMW). The exterior tube 50 and barriers 58, 52 may be formed as a unitary part and connected to surround the the injection line 34, or the parts may be co-forming with the flexible injection line. Alternatively, the interior barrier 58 may be formed separately from the exterior barrier 52 and attached to the injection line 34. The puncture resistant exterior tube 50 prevents the patient from piercing the injection line with a hypodermic syringe and injecting fluids into the injection line.

As shown in FIG. 4, a gap 51 is defined between the exterior barrier 52 and the interior barrier 58 which receives the end wall 46. The exterior tube 50 prevents access to the injection line 34 where it runs between the picc line base 32 and the V-shaped entry slot 47 of lock 22, and the exterior barrier 52 prevents access to the interior of the lock housing 43 through the entry slot 47, while the interior barrier 58 restricts the injection line from being pulled out of the housing 43.

An alternative embodiment catheter assembly 69 is shown in FIGS. 6 and 7 which has an injection line 70 with a puncture resistant exterior tube 72 formed as a unitary piece with an exterior barrier 74 and an interior barrier 76. The interior barrier 76 is connected to the exterior barrier 74 by a molded web 78. The web 78 tapers from a region that is narrower than the injection line 70 which is below the injection line, to a region that is wider than the injection line above the injection line. The web 78 is thus shaped to fit within the V-shaped entry slot 47 of the housing 43 of the lock 22. Thus when the catheter assembly 69 is inserted into the lock 22, the V-shaped entry slot can clamp the flexible injection line, without any interference from the web 78. As illustrated in FIG. 7, the exterior barrier 74 may be a full disc and the interior barrier 76 may be less than a full disc, with its upper region truncated so that the lid 45 of the lock can be slid over the interior barrier 76 and access be blocked by the exterior barrier.

Alternatively, as shown in FIG. 8, a catheter assembly 80 has an exterior tube 82 overlying an injection line 83 with an exterior barrier 84, and an interior barrier 86. The exterior barrier 84 and interior barrier 86 are connected by a cylindrical connector 88 which may be the same size as the exterior tube 82. The catheter assembly 80 may be used where a clamping of the injection line 83 at the inlet slot is not required. In such an embodiment, the exterior tube, exterior barrier, interior barrier and connector 88 are integrally formed, and the slot need not be V-shaped.

The catheter assemblies 20 thus are readily used with catheter locks 22 to effectively provide a tamper evident arrangement along the length of the injection line between the lock and the base 32.

The puncture resistance of the exterior tube or layer 50 may be such as to pass the ASTM 2016 F2878-10 Hypodermic Needle Test Puncture Test Standard, Level 5, which is incorporated herein by reference, which are specifically defines puncture resistance of materials to common hypodermic needles in 21-, 25-, 28-gauge. The level 5 test employs a force applies to hypodermic needles of at least 10 or more Newtons driven at a velocity of 500 mm/minute at a 90° angle to the test material to qualify a material as meeting the standard.

It is understood that the invention is not limited to the particular construction and arrangement of parts herein illustrated and described, but embraces all such modified forms thereof as come within the scope of the following claims.

I claim:

1. A catheter assembly for use with a catheter lock enclosure having a bottom part with an end wall which forma an entry slot, the catheter assembly comprising:
a flexible injection line extending from a picc line base for mounting to a patient;
an puncture resistant exterior tube which surrounds the injection line and extends from the picc line base to an exterior barrier extending radially outwardly from the exterior tube; and
an interior barrier extending radially outwardly from the injection line and spaced axially along the injection line from the exterior barrier, wherein a gap is defined between the interior barrier and the exterior barrier, such that the lock enclosure bottom part end wall is receivable within the gap to restrain the position of the catheter assembly with respect to the lock enclosure, and to restrict access to an interior of the catheter lock enclosure.

2. The catheter assembly of claim 1 where in the exterior barrier and the interior barrier are form a unitary part with the injection line.

3. The catheter assembly of claim 1 wherein injection line and the puncture resistant exterior tube are form a unitary tube so that together they are made of the same material.

4. The catheter assembly of claim 1 wherein injection line and the puncture resistant exterior tube are form a unitary tube with the injection line as co-extruded, bonded together, or the exterior tube is shrink fitted over the injection line.

5. The catheter assembly of claim 1 wherein the puncture resistant exterior tube terminates at the exterior barrier, and the in interior barrier is formed with or attached to the injection line.

6. The catheter assembly of claim 1 wherein the puncture resistant exterior tube meet the ASTM 2016 F2878-10 Hypodermic Needle Test Puncture Test Standard, Level 5.

7. The catheter assembly of claim 1 catheter lock enclosure end wall forms a V-shaped entry slot.

8. The catheter assembly and lock of claim wherein interior barrier has portion which extend axially along the injection line to fill portions of the V-shaped slot above and below the injection line.

9. A catheter assembly and lock for preventing the injection of fluids into a picc line comprising:
a lockable enclosure, portions of the lockable enclosure forming a opening:
a flexible injection line extending from a picc line base through the opening, so that a injection port connected to the flexible injection line is contained within the lockable enclosure;
an puncture resistant exterior tube which surrounds the injection line and extends from a picc line base to an exterior barrier extending radially outwardly from the exterior tube; and
an interior barrier extending radially outwardly from the injection line and spaced axially along the injection line from the exterior barrier, so that the flexible injection line passes through the opening between the interior barrier and the exterior barrier, such that the injection line is restrain from withdrawal from the respect to the lockable enclosure and the injection port is held within the lockable enclosure.

10. The catheter assembly and lock of claim 9 where in the exterior barrier and the interior barrier are form a unitary part with the injection line.

11. The catheter assembly and lock of claim 9 wherein injection line and the puncture resistant exterior tube are form a unitary tube so that together they are made of the same material.

12. The catheter assembly and lock of claim 9 wherein injection line and the puncture resistant exterior tube are form a unitary tube with the injection line: as co-extruded, bonded together, or wherein exterior tube is shrink fitted over the injection line.

13. The catheter assembly and lock of claim 9 wherein the puncture resistant exterior tube terminates at the exterior barrier, and the in interior barrier is formed with or attached to the injection line.

14. The catheter assembly and lock of claim 9 wherein the puncture resistant exterior tube meet the ASTM 2016 F2878-10 Hypodermic Needle Test Puncture Test Standard, Level 5.

15. The catheter assembly and lock of claim 9 where the lockable enclosure is form of two parts and wherein the opening is partly formed in each of the two parts so that when the two parts are separated the lockable enclosure is completely separable from the injection line and the injection port.

16. The catheter assembly and lock of claim 15 wherein the one of the two parts has portion forming a V-shaped slot which is part of the opening and is arranged to clamp the injection line.

17. The catheter assembly and lock of claim 16 wherein the interior barrier has portion which extend axially along the injection line to fill portions of the V-shaped slot above and below the injection line.

18. A catheter assembly for use with a catheter lock enclosure having portions forming an opening in to the catheter lock enclosure, catheter assembly comprising:
a puncture resistant flexible injection line which meets ASTM 2016 F2878-10 Hypodermic Needle Test Puncture Test Standard, Level 5, said injection line extending from a picc line base for mounting to a patient;
wherein the injection line has exterior barrier extending radially outwardly from the injection line and an interior barrier extending radially outwardly from the injection line and spaced axially along the injection line from the exterior barrier, wherein a gap is defined between the interior barrier and the exterior barrier, such that the catheter lock enclosure opening is receivable within the gap with the exterior barrier out side of the catheter lock enclosure, and the interior barrier captured within the catheter lock enclosure, to restrain the position of the catheter assembly with respect to the catheter lock enclosure, and to restrict access to an interior of the catheter lock enclosure when the catheter lock enclosure is locked.

19. The catheter assembly and lock of claim 18 where the catheter lock enclosure is form of two parts and wherein the opening is partly formed in each of the two parts so that when the two parts are separated the lockable enclosure is completely separable from the injection line and the injection port.

20. The catheter assembly and lock of claim 19 wherein the one of the two parts has portion forming a V-shaped slot which is part of the opening and is arranged to clamp the injection line; and
wherein the interior barrier has portion which extend axially along the injection line to fill portions of the V-shaped slot above and below the injection line.
